(19) United States
(12) Patent Application Publication
(10) Pub. No.: US 2024/0428915 A1
(43) Pub. Date: Dec. 26, 2024

(54) Methods for Treating and Diagnosing Risk of Renal Allograft Fibrosis and Rejection

(71) Applicant: Icahn School of Medicine at Mount Sinai, New York, NY (US)

(72) Inventors: Barbara Murphy, New York, NY (US); Weijia Zhang, New York, NY (US)

(21) Appl. No.: 18/690,321

(22) PCT Filed: Sep. 14, 2022
(86) PCT No.: PCT/US2022/043423

§ 371 Date: Mar. 8, 2024

Related U.S. Application Data
(60) Provisional application No. 63/245,115, filed on Sep. 16, 2021.

(51) Int. Cl. G16H 20/10; A61K 31/343; A61K 31/436; A61K 31/4412; A61K 31/52; A61K 31/5377; A61K 31/573; A61K 38/18; A61K 38/22; C12N 15/10; C12Q 1/6883; G16H 50/30

(52) U.S. Cl. CPC G16H 20/10; A61K 31/343; A61K 31/436; A61K 31/4412; A61K 31/52; A61K 31/5377; A61K 31/573; A61K 38/1833; A61K 38/1875; A61K 38/2221; C12N 15/1096; C12Q 1/6883; G16H 50/30; C12Q 2600/158

(57) ABSTRACT

A method for identifying a renal allograft recipient's risk for developing fibrosis of the allograft and allograft loss is disclosed. The method includes identifying the allograft recipient as being at risk for fibrosis of the allograft and allograft loss when the expression level of one or more genes in a preselected gene signature set is altered relative to the expression level of the same one or more genes in a control blood specimen is indicative of the allograft recipient's risk of developing fibrosis of the allograft and allograft loss.

### GOVERNMENT GRANT CLAUSE

This invention was made with government support under grant No. 5U01AI070107 awarded by The National Institutes of Health. The government has certain rights in the invention.

### TECHNICAL FIELD

This invention relates to the field of molecular biology, and more particularly to detecting mRNA molecular signatures. More particularly, this invention relates to methods for diagnosing a renal allograft recipient's risk for developing fibrosis of the allograft and allograft loss. The methods comprise analyzing the blood of renal allograft recipients by determining the expression level of a preselected gene signature set comprising 9 genes in order to identify and treat such patients. The difference of expression value of 9 genes (e.g. read counts of genes from next generation sequencing technology) between the patients that do and do not develop fibrosis are summarized to derived a statistical model from which a cumulative risk score for fibrosis of the allograft and allograft loss can be determined for each patient.

### BACKGROUND

Progressive renal fibrosis leading to decline in renal function remains the predominant cause of renal allograft loss. Current methodologies based on clinical and pathological parameters fail to identify grafts at risk for loss prior to the development of irreversible injury. Such tests usually require obtaining a biopsy specimen from the patient. Often by the time rejection is recognized it is too late to do anything. An increase in serum creatinine or an increase of protein in the urine may be warnings of rejection but are not entirely predictive. Furthermore, the collection and assaying of patient biopsy samples is time-consuming and expensive.

Thus, there remains a need for improved diagnostic methods for predicting a renal allograft recipient's risk for developing fibrosis of the allograft and allograft loss.

### SUMMARY OF THE INVENTION

Disclosed herein is a method for identifying a renal allograft recipient's risk for developing fibrosis of the allograft and allograft loss. The method comprises identifying the allograft recipient as being at risk for fibrosis of the allograft and allograft loss when the expression level of one or more genes in a preselected gene signature set is altered relative to the expression level of the same one or more genes in a control blood specimen is indicative of the allograft recipient's risk of developing fibrosis of the allograft and allograft loss.

In one aspect, the present invention provides a method for treating a human renal allograft recipient at risk for developing fibrosis of the allograft comprising the steps of: (a) selecting a human renal allograft recipient identified as being at risk for fibrosis of the allograft based on detected expression levels of mRNAs encoded by a preselected gene signature set which are higher than the expression levels of a control blood specimen obtained from a second renal allograft recipient that did not develop fibrosis of the allograft, said expression levels obtained by synthesizing cDNA from mRNA isolated from a blood specimen obtained from said renal allograft recipient, and detecting the expression levels of mRNAs encoded by a gene signature InNLRC5 and KIAA1683 in the cDNA; and administering to the selected human renal allograft recipient an effective amount of an anti-fibrotic agent, an anti-rejection drug, or both.

In another aspect, the method comprises applying the expression levels determined in the allograft recipient's sample to a penalized logistic regression fitting model to identify the allograft recipient's risk of fibrosis.

In a further aspect of the invention, the penalized logistic regression fitting model utilizes the formula: r=−(log₁₀(p₁)*g₁+log₁₀(p₂)*g₂+ . . . +log₁₀(pᵢ)*gᵢ+ . . . +log₁₀(p₉)*g₉), where pᵢ is the significance p value of t-test on expression values for gene i (i=1 . . . 9) between the patients with and without development of fibrosis in the training set, gᵢ is a logic number for gene i (i=1 . . . 9), 1 (if the expression value of gene i is greater than the median expression value of the fibrosis group of the training set for an upregulated gene or if the expression value of gene i is less than the median expression value of the fibrosis group of the training set for a downregulated gene), or −1 (if the expression value of gene i is less than the median value of the non-fibrosis group of the training set for an upregulated gene or if the expression value of gene i is greater than the median value of the non-fibrosis group of the training set for a downregulated gene), or 0 (if the expression value of gene i is between the median values of the fibrosis and the non-fibrosis groups of the training set).

In a still further aspect, the anti-rejection drug is an immunosuppressive or anti-proliferative agent.

In another aspect the immunosuppressive agent is a member selected from the group consisting of a mycophenolate mofetil (MMF), prednisone, Mycophenolate Sodium and Azathioprine.

In another aspect, the anti-fibrosis drug is a member selected from the group consisting of Pirfenidone, relaxin, Bone morphogenetic protein 7 (BMP-7) and Hepatic growth factor (HGF) 6.

In another aspect, detecting the expression levels of the mRNAs-comprises performing an assay that is a member selected from the group consisting of qPCR analysis, Nanostring analysis and TREx analysis.

Another aspect of the invention comprises modifying the immunosuppression regimen of an allograft recipient identified as being at risk for fibrosis of the allograft.

In another aspect, modifying the immunosuppression regimen comprises administering to the allograft recipient an effective amount of an anti-rejection drug selected from the group consisting of Belatacept, rapamycin and Mycophenolate Mofetil.

In another aspect, modifying the immunosuppression regimen comprises administering to the allograft recipient an anti-fibrosis drug selected from the group consisting of Pirfenidone, relaxin, Bone morphogenetic protein 7 (BMP-7) and Hepatic growth factor (HGF) 6.

In another aspect, the present invention provides a method for selecting a human renal allograft recipient for treatment to reduce the risk for fibrosis of the allograft comprising the steps of: (a) detecting expression levels of mRNAs encoded by genes in a gene signature set in a blood sample obtained from the renal allograft recipient, wherein said genes are NTSR1, TSPAN14, CCR3, SEC22C, FCER1G, YBEY, CLDN18, NLRC5 and KIAA1683, and (b) identifying the human renal allograft recipient as being at risk for fibrosis of the allograft when the expression levels the mRNAs encoded by the genes in the gene signature set are higher than the expression levels of the mRNAs encoded by same genes in a control.

Another aspect of the invention comprises determining the expression levels by synthesizing cDNA from mRNA isolated from a blood specimen obtained from said renal allograft recipient, and detecting the mRNA expression levels of the genes in the gene signature set in the cDNA.

Another aspect the present invention comprises applying the mRNA expression levels determined in the recipient's blood sample to a penalized logistic regression fitting model.

Another aspect of the invention comprises detecting the expression levels of said mRNAs with an assay that is a member selected from the group consisting of qPCR analysis, Nanostring analysis and TREx analysis.

In yet another aspect, the present invention provides a method for identifying a renal allograft recipient at risk for fibrosis of the allograft and allograft loss, comprising the steps of—(a) detecting expression levels of mRNAs encoded by a gene signature set comprising the genes NTSR1, TSPAN14, CCR3, SEC22C, FCER1G, YBEY, CLDN18, NLRC5 and KIAA1683 from a blood sample obtained from the renal allograft recipient, and—(b) identifying the human renal allograft recipient as being at risk for fibrosis of the allograft and allograft loss when the expression levels of the mRNAs encoded by the genes in the signature set are higher than the expression levels of the mRNAs encoded by the gene signature set genes in a control.

Another aspect of the invention comprises administering to the allograft recipient identified as being at risk for fibrosis of the allograft and allograft loss an effective amount of an anti-rejection drug, an immunosuppressive agent, an anti-fibrotic agent or combinations thereof.

Another aspect of the invention comprises applying the expression levels determined in the allograft recipient's sample to a penalized logistic regression fitting model to identify the allograft recipient's risk of fibrosis.

In yet another aspect, the present invention provides a method for identifying a renal allograft recipient at risk for fibrosis of the allograft and allograft loss, comprising the steps of (a) detecting expression levels of mRNAs encoded by a gene signature set comprising the genes NTSR1, TSPAN14, CCR3, SEC22C, FCER1G, YBEY, CLDN18, NLRC5 and KIAA1683 from a blood sample obtained from the renal allograft recipient, and (b) identifying the human renal allograft recipient as being at risk for fibrosis of the allograft and allograft loss when the expression levels of the mRNAs encoded by the gene signature set are higher than the expression levels of the mRNAs encoded by the gene signature set genes in a control.

Another aspect of the invention comprises administering to the allograft recipient identified as being at risk for fibrosis of the allograft and allograft loss an effective amount of an anti-rejection drug, an immune suppressive agent, an anti-fibrotic agent or combinations thereof.

Another aspect of the invention comprises applying the expression levels determined in the allograft recipient's sample to a penalized logistic regression fitting model to identify the allograft recipient's risk of fibrosis.

In another aspect the present invention provides a method for treating a human renal allograft recipient identified as being at risk for fibrosis of the allograft based on detected expression levels of mRNAs encoded by a preselected gene signature set which are higher than the mRNA expression levels of the gene signature set in a control, said expression levels obtained by synthesizing cDNA from mRNA isolated from a blood specimen obtained from said renal allograft recipient, and detecting the expression levels of mRNAs encoded by a gene signature set comprising the genes NTSR1, TSPAN14, CCR3, SEC22C, FCER1G, YBEY, CLDN18, NLRC5 and KIAA1683 in the cDNA; and (b) administering to the selected human renal allograft recipient an effective amount of an anti-fibrotic agent, an anti-rejection drug, or both.

In another aspect of the invention the control level is computed based on the mRNA expression levels of the gene signature set.

These and other aspects and embodiments of the present invention will be apparent to those of ordinary skill in the art from the description, drawings and claims.

### BRIEF DESCRIPTION OF DRAWINGS

FIG. 1 shows the data set selection for the Discovery Set (N=55, 20 vs 35), Validation Set 1 (N=30, 10 vs 20) and Validation Set 2 (N=48. 31 vs 17)

FIG. 2: Workflow for the discovery of the 9 member gene signature set of the present invention.

FIG. 3: Building the statistical model for estimating the risk score in the training set (N=55): a) The ROC curve of prediction of fibrosis with 9 geneset in the training set (AUC=0.9) b) The dot plot of the risk scores in the training set. At tertile cutoff {−4.98, 1.57}, PPV and NPV is 0.88 and 1, respectively.

FIG. 4: Validation of the statistical model to estimating the risk score in the validation set 1 (V1: N=30): a) The ROC curve of prediction of fibrosis with 9 geneset in V1 (AUC=0.79); b) The dot plot of the risk scores in the training set. At the same tertile cutoff defined in the training set, PPV and NPV is 0.85 and 0.81, respectively.

### DETAILED DESCRIPTION

### Overview

The present invention is directed to methods for diagnosing a renal allograft recipient's risk for developing fibrosis of the allograft and allograft loss. Fibrosis can result in loss of the allograft. The methods described herein are useful for identifying whether a renal allograft recipient is at risk for developing fibrosis of the allograft and allograft loss. Stated another way, the methods described herein are useful for determining the probability a renal allograft recipient risk for developing fibrosis of the allograft and allograft loss. The methods relying on differences in the relative amounts (e.g., expression level) of mRNA obtained from the recipient, wherein the probability for developing fibrosis of the allograft and allograft loss, is determined as described herein.

The method of the present invention comprises the determination of an alteration in the levels of expression of a 9 gene signature set in a test sample from an allograft recipient. In particularly preferred embodiment, the gene signature set comprises the genes NTSR1, TSPAN14, CCR3, SEC22C, FCER1G, YBEY, CLDN18, NLRC5 and KIAA1683. An increase in the expression levels of one or more of the genes in the gene signature set relative to the expression levels of the same one or more genes in a control sample is indicative of the allograft recipient's risk of developing fibrosis of the allograft and allograft loss.

Pursuant to the present invention, an increased risk for developing fibrosis of the allograft and allograft loss corresponds to a 12-month Chronic Allograft Damage Index CADI-12 score of 1 or greater. The CADI score for a kidney transplant is based on individual component scores for a) diffuse or focal inflammation, b) fibrosis in the allograft interstitium, c) increase in mesangial matrix, d) sclerosis in glomeruli, e) intimal proliferation, and f) tubular atrophy. Each individual parameter is scored from 0 to 3 as described in the literature (Yilmaz et al., 2003, Journal of the American Society of Nephrology: JASN. 14:773-779).

In practicing the present invention, identifying an allograft recipient's risk comprises calculating the recipient's risk (r) by applying the expression levels determined in the recipient's sample to a cumulative statistical model summarizing the difference of 9 genes between the recipient and the control. The formula employed for the risk assessment is r=−(log₁₀(p₁)*g₁+log₁₀(p₂)*g₂+ . . . +log₁₀(pᵢ)*gᵢ+ . . . +log₁₀(p₉)*g₉), where pᵢ is the significance p value of t-test on expression values for gene i (i=1 . . . 9) between the patients with and without development of fibrosis in the training set, gᵢ is a logic number for gene i (i=1 . . . 9), 1 (if the expression value of gene i is greater than the median expression value of the fibrosis group of the training set for an upregulated gene or if the expression value of gene i is less than the median expression value of the fibrosis group of the training set for a downregulated gene), or −1 (if the expression value of gene i is less than the median value of the non-fibrosis group of the training set for an upregulated gene or if the expression value of gene i is greater than the median value of the non-fibrosis group of the training set for a downregulated gene), or 0 (if the expression value of gene i is between the median values of the fibrosis and the non-fibrosis groups of the training set).

The weighted cumulative score (r) can be used as a risk score for development of fibrosis for each patient. If the risk score of the patient is higher than the tertile expression value cutoffs defined from the training dataset, then the patient is at risk for development of fibrosis. In certain embodiments, the method further comprises administration of a treatment based on the diagnosis.

The assay method disclosed herein is a blood-based assay that avoids the need for biopsy specimens. Allograft recipients can be monitored using the assays disclosed herein at the time of transplant, early post-transplant and periodically thereafter. When an allograft recipient is determined to be at increased risk for developing fibrosis of the allograft and for allograft loss, the allograft recipient can be treated, e.g., through modification of the allograft recipient's immunosuppression regimen, such as, for example administering, discontinuing administration or adjusting the dosage of an immunosuppressive drug (e.g., anti-rejection drugs), or by administering one or more anti-fibrosis agents.

The method disclosed herein addresses the need for improved methods for identifying a renal allograft recipient at risk for developing fibrosis of the allograft and allograft loss, and provides a blood based assay that is easily administered repetitively to transplant recipients. Renal transplant patients are examined by their physician very frequently post transplantation, in most instances twice per week for the first month after transplant moving to weekly and then every other week, getting to monthly after 4 to 5 months, with time intervals between visits gradually increasing thereafter. During this time, the patients' renal function and the immunosuppression levels are monitored. Steroids are typically tapered to 5 mg by 3 months post-surgery and the tacrolimus (a drug that suppresses the immune system and is used to prevent rejection of transplanted organs) levels are gradually reduced to a steady level by 6-12 months if the post-transplant course has no complications and the patient is not at high immunological risk. The expression profiles described below can be employed as a standard test to be performed at the time of a clinical visit. A positive test result (i.e. if the expression levels of the nine gene signature set are increased relative to a control level for the same signature set indicates that the allograft recipient is at increased risk for developing fibrosis of the allograft and allograft loss, and would be treated by increasing modifying the patient's immunosuppressive dosing regimen and/or by administering anti-fibrosis drugs. Repeat testing (which can be done economically since the assay is preferably a blood based test) will guide the continued modifications, if any, to the patient's immunosuppressive dosing regimen.

Pursuant to the present invention, mRNA profiling identified a gene signature set for prediction of development of renal allograft fibrosis and allograft loss. It was discovered, in particular, that a nine gene signature set comprising the genes NTSR1, TSPAN14, CCR3, SEC22C, FCER1G, YBEY, CLDN18, NLRC5, KIAA1683t, can be used to identify an allograft recipient at risk for developing fibrosis of the allograft and allograft loss with a high predictive value. In allograft recipients who had a high CADI (e.g., CADI-12 score), the four out of nine genes in the gene signature set were upregulated and five genes were downregulated (Table 1). Thus, gene signatures have been developed based on this data, which identifies a patient as at risk of developing fibrosis of the allograft and allograft loss. Alternatively, the expression levels of any individual gene in the gene signature set can be determined to assess the allograft recipient's risk of fibrosis and allograft loss.

Particularly preferred individual genes for use in the present methods for treatment include, e.g., the gene signature set comprising the genes NTSR1, TSPAN14, CCR3, SEC22C, FCER1G, YBEY, CLDN18, NLRC5, KIAA1683.

In other embodiments, an allograft recipient's expression levels of the nine gene signature set are compared to a reference value or reference set (control) for the genes and the relative risk of the allograft recipient is assessed based on statistical analysis.

According to some embodiments, the method comprises determining the expression level (i.e., determining the level, measuring the amount, or measuring the level) of each (i.e., all) genes within a panel of nine genes in the gene signature set.

In other embodiments, the genes are analyzed collectively, and the relative risk for developing fibrosis of the allograft and allograft loss is assessed based on comparing the gene profile of the allograft recipient to a reference profile (e.g., derived from or based on the levels of a cohort of allograft recipients who are known to not be at risk for developing fibrosis of an allograft and allograft loss), wherein the comparison includes considering the expression profile of the nine gene signature set disclosed herein.

### Definitions

As used herein, an allograft recipient who is at “risk” or “increased risk” of developing fibrosis of the allograft and allograft loss is significantly more likely to develop fibrosis and allograft failure, without intervention.

In some embodiments, detection and quantification of mRNA expression requires isolation of nucleic acids from a sample, such as blood, plasma, a cell or a tissue. Nucleic acids, including RNA and specifically mRNA, can be isolated using any suitable technique known in the art. For example, phenol-based extraction is a common method for isolation of RNA. Phenol-based reagents contain a combination of denaturants and RNAase inhibitors for cell and tissue disruption and subsequent separation of RNA from contaminants. In addition, extraction procedures such as those using TRIZOL™ or TRI REAGENT™, will purify all RNAs, large and small, and are efficient methods for isolating total RNA from biological samples that contain mRNAs. Extraction procedures such as those using the QIAGEN-ALL prep kit are also contemplated.

PCR is commonly used for the purpose of determining whether a genetic sequence is present in a sample, and if it is present, the number of copies in the sample. Any method of PCR that can determine the expression of a nucleic acid molecule, including an mRNA, falls within the scope of the present invention. There are several variations of the qRT-PCR method that are well known to those of ordinary skill in the art.

As used herein, the term “about” or “approximately” usually means within an acceptable error range for the type of value and method of measurement. For example, it can mean within 20%, more preferably within 10%, and most preferably still within 5% of a given value or range. Alternatively, especially in biological systems, the term “about” means within about a log (i.e., an order of magnitude) preferably within a factor of two of a given value.

As used herein, “obtain” or “obtaining” can be any means whereby one comes into possession of the sample by “direct” or “indirect” means. Directly obtaining a sample means performing a process (e.g., performing a physical method such as extraction) to obtain the sample. Indirectly obtaining a sample refers to receiving the sample from another party or source (e.g., a third party laboratory that directly acquired the sample). Directly obtaining a sample includes performing a process that includes a physical change in a physical substance, e.g., a starting material, such as a blood, e.g., blood that was previously isolated from a patient. Thus, obtain is used to mean collection and/or removal of the sample from the subject. Furthermore, “obtain” is also used to mean where one receives the sample from another who was in possession of the sample previously.

As used herein, “determining the level of expression,” “determining the expression level” or “detecting the level of expression” as in, for example, “determining the expression level of mRNA” refers to quantifying the amount of an mRNA present in a sample. Detecting expression of the specific mRNA, can be achieved using any method known in the art or described herein. Typically, mRNA detection methods involve sequence specific detection, such as by RT-PCR. mRNA-specific primers and probes can be designed using the precursor and mature mRNA nucleic acid sequences, which are known in the art.

As used herein, an “altered” level of expression of a mRNA compared to a reference level or control level is an at least 0.5-fold (e.g., at least: 1- 2-; 3-; 4-; 5-; 6-; 7-; 8-; 9-; 10-; 15-; 20-; 30-; 40-; 50-; 75-; 100-; 200-; 500-; 1,000-; 2000-; 5,000-; or 10,000-fold) altered level of expression of the mRNA. Pursuant to the present invention is understood that the alteration is an increased level of expression. Alternatively, altered expression level is defined as an increase in the risk probability score using parameters in the logistic regression model established from a training patient group, comparing the probability score to the cutoff derived from a training set.

The terms “increased”, “increase” or “up-regulated” are all used herein to generally mean an increase by a statistically significant amount; for the avoidance of any doubt, the terms “increased” or “increase” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 0.5-fold (e.g., at least: 1- 2-; 3-; 4-; 5-; 6-; 7-; 8-; 9-; 10-; 15-; 20-; 30-; 40-; 50-; 75-; 100-; 200-; 500-; 1,000-; 2000-; 5,000-; or 10,000-fold) or greater as compared to a reference level.

As used herein, the term “selectively targets”, e.g., in the context of a probe for detecting mRNA expression, means the targeting agent binds specifically to the target, and does not bind nonspecifically to other targets.

Throughout the application and in the appended claims, it should be understood and is intended to be understood that use of the terms “drug”, “medication”, “agent” and “therapeutic agent” are interchangeable expressions defining the same or similar entities.

A “drug” refers generally to a chemical compound, small molecule, or other biologic composition, such as an antisense compound, antibody, protease inhibitor, hormone, chemokine or cytokine, capable of inducing a desired therapeutic or prophylactic effect when properly administered to a subject.

As used herein, “treating” or “treatment” of a state, disorder or condition includes: (1) preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition (e.g., fibrosis of a renal allograft and/or allograft loss); and/or (2) inhibiting the state, disorder or condition, i.e., arresting, reducing or delaying the development of the disease or a relapse thereof (in case of maintenance treatment) or at least one clinical or sub-clinical symptom thereof; and/or (3) relieving the disease, i.e., causing regression of the state, disorder or condition or at least one of its clinical or sub-clinical symptoms; and/or (4) causing a decrease in the severity of one or more symptoms of the disease. The benefit to a subject to be treated is either statistically significant or at least perceptible to the patient or to the physician.

As used herein, the term “inhibiting” of disease or condition (e.g., fibrosis of a renal allograft and/or allograft loss) means for example, to stop the development of one or more symptoms of a disease in a subject before they occur or are detectable, e.g., by the patient or the patient's doctor. Preferably, the disease or condition does not develop at all, i.e., no symptoms of the disease are detectable. However, it can also result in delaying or slowing of the development of one or more symptoms of the disease. Alternatively, or in addition, it can result in the decreasing of the severity of one or more subsequently developed symptoms.

As used herein “combination therapy” means the treatment of a subject in need of treatment with a certain composition or drug in which the subject is treated or given one or more other compositions or drugs for the disease in conjunction with the first and/or in conjunction with one or more other therapies, such as, e.g., an immunosuppressive therapy or other anti-rejection therapy. Such combination therapy can be sequential therapy wherein the patient is treated first with one treatment modality (e.g., drug or therapy), and then the other (e.g., drug or therapy), and so on, or all drugs and/or therapies can be administered simultaneously. In either case, these drugs and/or therapies are said to be “co-administered.” It is to be understood that “co-administered” does not necessarily mean that the drugs and/or therapies are administered in a combined form (i.e., they may be administered separately or together to the same or different sites at the same or different times).

The term “pharmaceutically acceptable derivative” as used herein means any pharmaceutically acceptable salt, solvate or prodrug, e.g., ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5th Edition, Vol. 1: Principles and Practice, which is incorporated herein by reference to the extent of teaching such derivatives. Pharmaceutically acceptable derivatives include salts, solvates, esters, carbamates, and/or phosphate esters.

As used herein the terms “therapeutically effective” and “effective amount”, used interchangeably, applied to a dose or amount refer to a quantity of a composition, compound or pharmaceutical formulation that is sufficient to result in a desired activity upon administration to an animal in need thereof. Within the context of the present invention, the term “therapeutically effective” refers to that quantity of a composition, compound or pharmaceutical formulation that is sufficient to reduce or eliminate at least one symptom of a disease or condition specified herein, e.g., fibrosis of an allograft and/or allograft loss.

When a combination of active ingredients is administered, the effective amount of the combination may or may not include amounts of each ingredient that would have been effective if administered individually. The dosage of the therapeutic formulation will vary, depending upon the nature of the disease or condition, the patient's medical history, the frequency of administration, the manner of administration, the clearance of the agent from the host, and the like. The initial dose may be larger, followed by smaller maintenance doses. The dose may be administered, e.g., weekly, biweekly, daily, semi-weekly, etc., to maintain an effective dosage level.

Therapeutically effective dosages can be determined stepwise by combinations of approaches such as (i) characterization of effective doses of the composition or compound in in vitro cell culture assays using tumor cell growth and/or survival as a readout followed by (ii) characterization in animal studies using tumor growth inhibition and/or animal survival as a readout, followed by (iii) characterization in human trials using decreased fibrosis and/or decreased allograft rejection as a readout.

### Diagnostic Methods

The present invention relates to methods useful for identifying (e.g., clinical evaluation, diagnosis, classification, prediction, profiling) an allograft recipient at risk for developing fibrosis of the allograft and/or allograft loss based on the levels or occurrence of certain analytes (e.g., mRNAs).

In one embodiment, there is provided a method of assessing when an allograft recipient has higher than normal risk for developing fibrosis of the allograft and/or allograft loss, comprising the steps of comparing the level of expression of one or more genes in a nine gene signature set in a sample with the expression level of the same one or more genes in a control, e.g., a sample from a healthy individual.

As used herein, levels refer to the amount or concentration of an analyte in a sample (e.g., a plasma or serum sample) or subject. Whereas, occurrence refers to the presence or absence of a detectable analyte in a sample. Thus, level is a continuous indicator of amount, whereas occurrence is a binary indicator of an analyte. In some cases, an occurrence may be determined using a threshold level above which a biomarker is present and below which a biomarker is absent.

The nine gene signature set described herein is particularly useful for identifying (e.g., assessing or evaluating) an allograft recipient at risk for developing fibrosis of the allograft and/or allograft loss. Moreover, the methods described herein are useful for identifying an allograft recipient at risk for developing fibrosis of the allograft and/or allograft loss. As used herein, identifying includes both diagnosing and aiding in diagnosing. Thus, other diagnostic criteria may be evaluated in conjunction with the results of the methods in order to make a diagnosis.

According to some embodiments, the method comprises determining the expression level (i.e., determining the level, measuring the amount, or measuring the level) of each (i.e., all) genes within a panel of nine genes in the gene signature set.

In some embodiments, the methods disclosed herein comprise comparing expression levels or occurrences to a reference. The reference can take on a variety of forms. In some cases, the reference comprises predetermined values for the plurality of gene products (e.g., each of the plurality of mRNAs). The predetermined value can take a variety of forms. It can be a level or occurrence of an analyte obtained from an allograft recipient previously diagnosed as being at risk for fibrosis of the allograft and allograft loss, or obtained from an allograft recipient known not to be at risk for fibrosis of the allograft and allograft loss (e.g., an asymptomatic subject). It can be a level or occurrence obtained from a subject having not received a renal allograft. It can be a level or occurrence in the same recipient, e.g., at a different time point.

A predetermined value that represents a level(s) of an analyte is referred to herein as a predetermined level. A predetermined level can be single cut-off value, such as a median or mean. It can be a range of cut-off (or threshold) values, such as a confidence interval. It can be established based upon comparative groups, such as where the risk in one defined group is a single fold higher, (e.g., approximately 2-fold, 4-fold, 8-fold, 16-fold or more) than the risk in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk. Moreover, the reference could be a calculated reference, most preferably the average or median, for the relative or absolute amount of an analyte of a population of individuals comprising the subject to be investigated.

The absolute or relative amounts of the analytes of said individuals of the population can be determined as specified elsewhere herein. How to calculate a suitable reference value, preferably, the average or median, is well known in the art. The population of subjects referred to before shall comprise a plurality of subjects, preferably, at least 5, 10, 50, 100, 1,000 subjects. It is to be understood that the subject to be diagnosed by the method of the present invention and the subjects of the said plurality of subjects are of the same species.

Subjects associated with predetermined values are typically referred to as control subjects (or controls). A control subject may or may not have received a renal allograft. In some cases it may be desirable that control subject is a symptomatic subject, and in other cases it may be desirable that a control subject is an asymptomatic subject.

A level in some embodiments may itself be a relative level that reflects a comparison of levels between two states. Relative levels that reflect a comparison (e.g., ratio, difference, logarithmic difference, percentage change, etc.) between two states (e.g., healthy and diseased) may be referred to as delta values. The use of relative levels is beneficial in some cases because, to an extent, they exclude measurement related variations (e.g., laboratory personnel, laboratories, measurements devices, reagent lots/preparations, assay kits, etc.). However, the invention is not so limited.

Expression levels and/or reference expression levels may be stored in a suitable data storage medium (e.g., a database) and are, thus, also available for future diagnoses. This also allows efficiently diagnosing prevalence for a disease because suitable reference results can be identified in the database once it has been confirmed (in the future) that the subject from which the corresponding reference sample was obtained did develop fibrosis of the allograft and/or experience allograft rejection.

As used herein a “database” comprises data collected (e.g., analyte and/or reference level information and/or patient information) on a suitable storage medium. Moreover, the database, may further comprise a database management system. The database management system is, preferably, a network-based, hierarchical or object-oriented database management system. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative of renal allograft rejection risk. Thus, if an identical or similar data set can be identified in the data collection, the test data set will be associated with renal allograft rejection risk.

Consequently, the information obtained from the data collection can be used to identify an allograft recipient at risk for developing fibrosis of the allograft and/or allograft loss or based on a test data set obtained from a subject. More preferably, the data collection comprises characteristic values of all analytes comprised by any one of the groups recited above.

Also provided are databases of gene expression/protein signatures of different transplant categories, e.g., AR, STA, NS and the like. The gene expression/protein signatures and databases thereof may be provided in a variety of media to facilitate their use (e.g., in a user-accessible/readable format).

“Media” refers to a manufacture that contains the expression profile information of the present invention. The databases of the present invention can be recorded on computer readable media, e.g. any medium that can be read and accessed directly by a user employing a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information.

“Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc. Thus, the subject expression profile databases are accessible by a user, i.e., the database files are saved in a user-readable format (e.g., a computer readable format).

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention, e.g., to and from a user. One format for an output means ranks expression profiles possessing varying degrees of similarity to a reference expression profile. Such presentation provides a skilled artisan with a ranking of similarities and identifies the degree of similarity contained in the test expression profile.

In a typical embodiment, a clinical lab will obtain the expression value using the patient's sample and send it to the patient's doctor. The doctor will then communicate this value to his web based service provider. The service provider will enter that value in the bioinformatics system which already has the co-efficiency for each gene of the preselected gene set and the cutoff from the cumulative risk score estimation model from a training set. The bioinformatics system will use this information to calculate the probability score for the patient. The calculated score will reflect the patient's risk status.

The present invention further provides for the communication of assay results or diagnoses or both to technicians, physicians or patients, for example. In certain embodiments, computers will be used to communicate assay results or diagnoses or both to interested parties, e.g., physicians and their patients over the internet or by means of a hardwired or wireless telephone network.

In some embodiments, the method disclosed herein further comprises modifying the recipient's clinical record to identify the recipient as being at risk for developing fibrosis of the allograft and/or allograft loss. The clinical record may be stored in any suitable data storage medium (e.g., a computer readable medium).

In some embodiments, a diagnosis based on the methods provided herein is communicated to the allograft recipient as soon as possible after the diagnosis is obtained. The diagnosis may be communicated to the recipient by the recipient's treating physician. Alternatively, the diagnosis may be sent to a recipient by email or communicated to the subject by phone. The diagnosis may be sent to a recipient by in the form of a report. A computer may be used to communicate the diagnosis by email or phone. In certain embodiments, the message containing results of a diagnostic test may be generated and delivered automatically to the recipient using a combination of computer hardware and software which will be familiar to artisans skilled in telecommunications.

Aspects of the present invention include computer program products for identifying a subject who has undergone a renal allograft and is at risk for developing fibrosis of the allograft and allograft loss, wherein the computer program product, when loaded onto a computer, is configured to employ a mRNA expression result from a sample derived from the subject to determining whether a subject who has undergone a renal allograft is at risk for developing fibrosis of the allograft and allograft loss wherein the gene expression result comprises expression data for the nine gene panel provided herein.

The present invention also provides kits for evaluating mRNA expression levels in a subject (e.g. a renal allograft recipient). The kits of the invention can take on a variety of forms. Typically, the kits will include reagents suitable for determining mRNA expression levels (e.g., those disclosed herein) in a sample. Optionally, the kits may contain one or more control samples. Also, the kits, in some cases, will include written information (indicia) providing a reference (e.g., predetermined values), wherein a comparison between the mRNA expression levels in the subject and the reference (predetermined values) is indicative of a clinical status.

In some cases, the kits comprise software useful for comparing mRNA expression levels or occurrences with a reference (e.g., a prediction model). Usually the software will be provided in a computer readable format such as a compact disc, but it also may be available for downloading via the internet. However, the kits are not so limited and other variations with will be apparent to one of ordinary skill in the art.

The present methods can also be used for selecting a treatment and/or determining a treatment plan for a subject, based on the occurrence or levels of mRNA (e.g., those disclosed herein). In some embodiments, using the methods disclosed herein, a health care provider (e.g., a physician) identifies a recipient as being at risk for developing fibrosis of the allograft and/or allograft loss, and, based on this identification the health care provider determines an adequate management plan for the subject.

In some embodiments, using the method disclosed herein, a health care provider (e.g., a physician) identifies a recipient as being at risk for developing fibrosis of the allograft and/or allograft loss based on the occurrence or levels of certain genes in a clinical sample obtained from the subject, and/or based on a classification of a clinical sample obtained from the subject. By way of this diagnosis the health care provider determines an adequate treatment or treatment plan for the subject as described herein. In some embodiments, the methods further include administering the treatment the subject.

An exemplary procedure describing the application of a nine gene panel as described herein for identifying a renal allograft recipient at risk for developing fibrosis of the allograft and allograft loss is provided as follows:
1) Selecting a training group: A group of kidney transplant patients with high and low risk of cases (total number N=˜100) will be carefully selected. The training group should have well-characterized demographics and clinical indications which have been reviewed by at least two pathologists.
2) Measuring expression of 9 mRNAs: Expression levels of mRNAs transcribed from the nine gene signature set isolated from the blood sample post-transplant of each patient in the training group will be measured by RT-PCR Nanostring or TREx technology. Use of these techniques is described in the examples below.
3) Establishing regression model and cut off: a cumulative statistical model summarizing the difference of 9 genes between case and the control will be built from the training set. The formula employed for the risk assessment is r=−(log₁₀(p₁)*g₁+log₁₀(p₂)*g₂+ . . . +log₁₀(pᵢ)*gᵢ+ . . . +log₁₀(p₉)*g₉), where pᵢ is the significance p value of t-test on expression values for gene i (i=1 . . . 9) between the patients with and without development of fibrosis in the training set, gᵢ is a logic number for gene i (i=1 . . . 9), 1 (if the expression value of gene i is greater than the median expression value of the fibrosis group of the training set for an upregulated gene or if the expression value of gene i is less than the median expression value of the fibrosis group of the training set for a downregulated gene), or −1 (if the expression value of gene i is less than the median value of the non-fibrosis group of the training set for an upregulated gene or if the expression value of gene i is greater than the median value of the non-fibrosis group of the training set for a downregulated gene), or 0 (if the expression value of gene i is between the median values of the fibrosis and the non-fibrosis groups of the training set). The weighted cumulative score (r) can be used as a risk score for development of fibrosis for each patient. If the risk score of the patient is higher than the tertile expression value cutoffs defined from the training dataset, then the patient is at risk for development of fibrosis.

Based on the probability score, the prediction statistics such as prediction AUC (area under the curve) of ROC (Receive operating characteristic) curve of true positive rate versus false positive, sensitivity/specificity, the positive values (PPV) and negative predictive values (NPV) will be determined. At a given specificity (90%), a probability score cut off will be established which best predicts the development of fibrosis. This may be a clear cut off into two groups in that if they are in the top group they have a high likelihood of developing fibrosis and the test is determined to be positive but if they are in the bottom they have a very low likelihood of developing fibrosis and the test is determined to be negative. The alternative is that patients may be broken in to tertiles based on their probability score determined as above. In this case if the patient is in (1) the top tertile they have a high likelihood of developing fibrosis and the test is determined to be positive; (2) they are in the second tertile or intermediate group their risk cannot be accurately determined; and (3) they are in the bottom they have a very low likelihood of developing fibrosis and the test is determined to be negative.

The coefficiency (log₁₀(pᵢ)), the median expression values of case and control groups and the cutoff derived from the training group will be entered and stored into a web-based bioinformatics system which can be accessed from clinical lab/doctor office via the internet.
4) Diagnostic criteria: For a new patient, the expression levels of the nine gene signature set will be measured by the same technology as used for the training set in the clinical lab.) By using a web-based bioinformatics system, the probability score will be calculated from formula using the parameters which are derived from the training set and the probability score will be compared to the cutoff to determine the likelihood of developing fibrosis. The clinical lab will send the testing results to the doctor where if the result for the sample is above the cutoff for high likelihood of fibrosis it will be reported as negative.
5) Treatment: If the tests indicates that the patient has high risk of developing fibrosis of the allograft and allograft loss can be treated, for example, and without limitation, by administering an anti-fibrosis drug to the allograft recipient or switching the immunosuppressive regimen.

In some embodiments, the treatment includes modification of the allograft recipient's immunosuppression regimen, such as, for example, by administering, discontinuing administration, or adjusting the dosage of one or more immunosuppressive drugs, including, for example, or one or more anti-rejection drugs.

Immunosuppression can be achieved with many different drugs, including steroids, targeted antibodies and CNIs, like tacrolimus. Non-limiting examples of CNIs include, e.g. calcineurin inhibitor (CNI), such as cyclosporine or tacrolimus, or a less fibrogenic immunosuppressive drug such as mycophenolate mofetil (MMF) or sirolimus. The main class of immunosuppressants is the calcineurin inhibitors (CNIs), which includes tacrolimus (Prograf® and Advagraf®/Astagraf XL (Astellas Pharma Inc.) and generics of Prograf®) and cyclosporine (Neoral® and Sandimmune® (Novartis AG) and generics). Steroids such as prednisone may also be administered to treat patients at risk for developing fibrosis of the allograft and allograft loss. Anti-proliferative agents such as Mycophenolate Mofetil, Mycophenolate Sodium and Azathioprine are also useful in such treatments. Of these, tacrolimus is one of the more potent in terms of suppressing the immune system. The anti-rejection drug Belatacept (Bristol Myers Squibb) may also be employed for treatment of patients at risk for rejection or fibrosis.

Allograft recipients identified as being at increased risk for developing fibrosis of the allograft and allograft loss can also be treated, for example, and without limitation, by administration of an anti-fibrosis drug or by modifying the allograft recipient's immunosuppression regimen. Thus, treatment of the allograft recipient may include administering, discontinuing administration, or adjusting the dosage of one or more anti-fibrosis drugs. In some aspects, the anti-fibrosis drug may include an anti-fibrotic agents such as, for example, Pirfenidone, relaxin, Bone morphogenetic protein 7 (BMP-7), and Hepatic growth factor (HGF) 6.

Administration of an angiotensin converting enzyme inhibitor (ACEI) such as Lisinopril, or angiotensin II receptor blockades such as losartan, to such patients is also within the scope of the present disclosure.

In some aspects, the method comprises switching immunosuppression from a calcineurin inhibitor to a drug which is not associated with the development of fibrosis such as the anti-rejection drug is Belatacept, rapamycin or Mycophenolate Mofetil.

In certain embodiments, kits are provided for determining a renal allograft recipient's risk of developing fibrosis of the allograft and allograft loss. In a non-limiting example, reagents for isolating mRNA, labeling mRNA and/or evaluating an mRNA population are included in a kit. The kit may further include reagents for creating or synthesizing gene probes. The kits will thus comprise, in suitable container means, an enzyme for labeling the mRNA by incorporating labeled nucleotide or unlabeled nucleotides that are subsequently labeled. It may also include one or more buffers, such as reaction buffer, labeling buffer, washing buffer, or a hybridization buffer, compounds for preparing the mRNA probes, and components for isolating mRNA.

For any kit embodiment, there can be nucleic acid molecules that contain a sequence that is identical or complementary to all or part of any of the sequences herein.

The above kits can include barcode probes that specifically hybridize to one or more of the genes NTSR1, TSPAN14, CCR3, SEC22C, FCER1G, YBEY, CLDN18, NLRC5, KIAA1683 (e.g., for use in Nanostring analysis). The kits can further contain one or more mRNA extraction reagents and/or annealing reagents.

In some embodiments, the kits will contain the primers for amplifying the genes NTSR1, TSPAN14, CCR3, SEC22C, FCER1G, YBEY, CLDN18, NLRC5, KIAA1683 and optionally comprising primers for amplifying control sequences, such as, for example, primers for amplifying beta actin (ACTB) and glyceraldehyde 3-phosphate dehydrogenase (GAPDH), 18S ribosomal RNA (for qPCR assays), and fragments thereof.

In other embodiment, a kit can contain an mRNA inhibitor (e.g., targeted to an mRNA that is upregulated in allograft recipients at high risk of developing fibrosis of the allograft and allograft loss (e.g., the genes NTSR1, TSPAN14, CCR3, SEC22C, FCER1G, YBEY, CLDN18, NLRC5, KIAA1683).

The kits, regardless of type, will generally comprise one or more containers into which the biological agents are placed and, preferably, suitably aliquotted. The components of the kits may be packaged either in aqueous media or in lyophilized form. The kits can also comprise one or more pharmaceutically acceptable excipients, diluents, and/or

Non-limiting examples of pharmaceutically acceptable excipients, diluents, and/or carriers include RNAase-free water, distilled water, buffered water, physiological saline, PBS, Ringer's solution, dextrose solution, reaction buffers, labeling buffers, washing buffers, and hybridization buffers.

The kits can also include instructions for employing the kit components as well the use of any other reagent not included in the kit. Instructions may include variations that can be implemented. It is contemplated that such reagents are embodiments of kits of the invention. Also, the kits are not limited to the particular items identified above and may include any reagent used for the manipulation or characterization of genes.

The kits contemplated herein can further contain one or more mRNA extraction reagents and/or reagents for cDNA synthesis.

The kits for therapeutic, prognostic, or diagnostic applications and such uses are contemplated. Also, in certain embodiments, control RNA or DNA can be included in the kit. The control RNA can be mRNA that can be used as a positive control for the diagnostic assays disclosed herein. A non-limiting list of housekeeping genes for use with the kits of the present invention is set forth in Example 6 below.

The present invention is described below in examples which are intended to further describe the invention without limiting the scope thereof.

### EXAMPLE 1: RNA SEQUENCING ASSAY: IDENTIFICATION OF 9-GENE SET AND ITS APPLICATION TO PREDICT FIBROSIS

### RNA Sequencing Assay Kit Includes

1) Illumina TruSeq mRNA Library Prep Kit
2) TruSeq RNA Single Indexes Set
3) QIAGEN RNeasyR Kit for extraction of high quality total RNA

### Methods for RNA Sequencing and Data Processing

Total RNA was extracted from whole blood drawn from kidney transplant recipients prior to transplantation (baseline) using QIAGEN RNeasy® Kit, and the libraries were generated with TruSeq mRNA Library Prep Kit and were further multiplexed with TruSeq RNA Single Indexes. The indexed libraries were sequenced on Illumina HiSeq4000 sequencer. The reads with good quality were firstly aligned to human reference databases including hg19 human genome, exon, splicing junction segment and contamination database including ribosome and mitochondria sequences using the BWA alignment algorithm. After filtering for reads mapped to contamination database, the reads that are uniquely aligned to the exon and splicing-junction segments with a maximal 2 mismatches for each transcript were then counted as the expression level for each corresponding transcript. The read counts were log2 transformed and normalized at an equal global median value in order to compare transcription levels across samples.

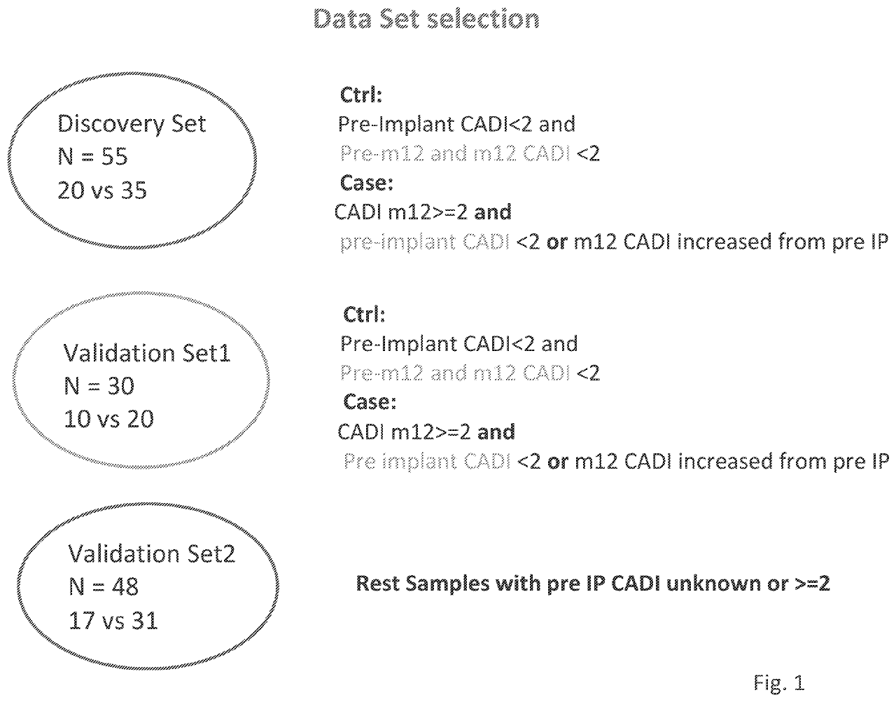

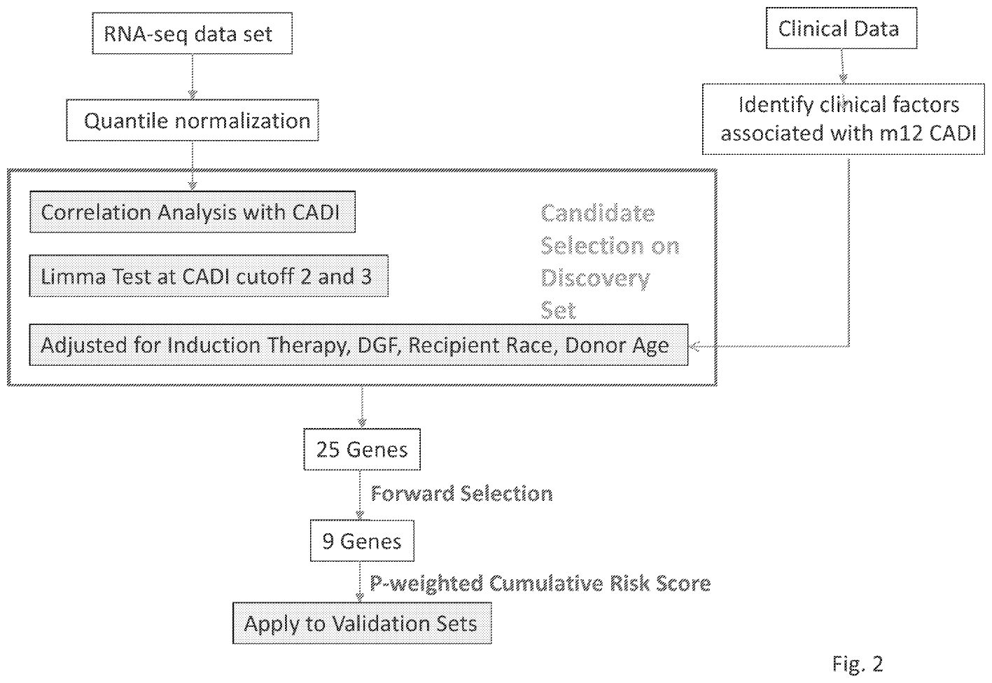

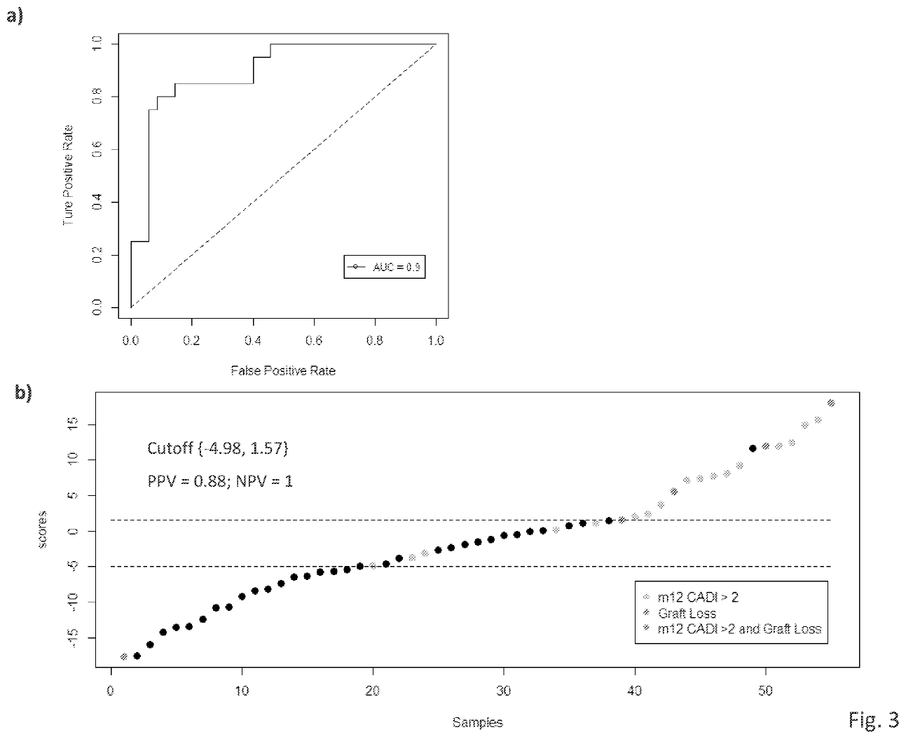

The results:
a) Patient cohort description: This study included a total of 133 patients with good quality baseline blood RNA that underwent RNA sequencing (FIG. 1), Out of the 133 patients, 85 patients had pre-implant good kidney with pre-implantation CADI<2 or fibrosis progression with CADI increase (m12 CADI>=2). Out of 85 patients with early surveillance biopsies, 55 were randomly selected to be used as the discovery set for the identification of the gene signature set for predicting fibrosis development diagnosed based on CADI score>=2 from pathology slides of kidney biopsies at 12 month after transplant The discovery and VI set with well-characterized fibrosis progression (Case group: CADI increase post-transplant) and non-progression (pre-imp CADI<2 and m12 CADI<2) non-fibrosis diagnosis was used to identify and validate the geneset and derive the p value and median expression of the fibrosis and non-fibrosis group for gene risk score calculation. The remaining 30 patients were used as the first validation cohort (V1). The 48 patients had a high pre-implantation donor kidney CADI score (pre-imp CADI>2) or no pathology grading were used as the second validation set 2 (V2).
b) Identification of the 9 gene set: The workflow for the 9 geneset identification is depicted in FIG. 2. Briefly, RNA sequencing was performed on the discovery set of 55 patients. After a series of read quality controls, mapping and normalization steps on the raw sequence reads (4), the normalized read counts were represented as expression values of the genes. The expression values for the fibrosis and the non-fibrosis groups in the discovery set (N=55) were compared by using the well-known LIMMA (5) test to identify differentially expressed genes (DEG) between the patients that developed and didn't develop fibrosis. The gene list was further filtered by adjustment for demographic and clinical factors, ending up with 25 genes. The difference of the expression values of the 25 DEGs between fibrosis and non-fibrosis groups were summarized to derive a cumulative risk score for the development of fibrosis and a forward section was iteratively applied to the 25 genes to determine the final 9 gene set with maximal predictive accuracy (AUC=0.9 FIG. 3a, Table 1). The formula employed for the risk assessment is r=−(log₁₀(p₁)*g₁+log₁₀(p₂)*g₂+ . . . +log₁₀(pᵢ)*gᵢ+ . . . +log₁₀(p₉)*g₉), where pᵢ is the significance p value of t-test on expression values for gene i (i=1 . . . 9) between the patients with and without development of fibrosis in the training set, gᵢ is a logic number for gene i (i=1 . . . 9), 1 (if the expression value of gene i is greater than the median expression value of the fibrosis group of the training set for an upregulated gene or if the expression value of gene i is less than the median expression value of the fibrosis group of the training set for a downregulated gene), or −1 (if the expression value of gene i is less than the median value of the non-fibrosis group of the training set for an upregulated gene or if the expression value of gene i is greater than the median value of the non-fibrosis group of the training set for a downregulated gene), or 0 (if the expression value of gene i is between the median values of the fibrosis and the non-fibrosis groups of the training set).

TABLE 1
The 9 gene set for the prediction of fibrosis development

| Symbol | RefSeq | Name | p | Log2Rat |
|---|---|---|---|---|
| CLDN18 | NM_016369 | claudin 18 | 0.0056 | 1.34 |
| KIAA1683 | NM_001145304 | KIAA1683 | 0.0005 | 0.81 |
| CCR33 | NM_001164680 | chemokine (C-C motif) receptor 3 | 0.0036 | 0.81 |
| YBEY | NM_058181 | ybeY metallopeptidase (putative) | 0.0083 | 0.38 |
| TSPAN14 | NM_030927 | tetraspanin 14 | 0.0184 | −0.14 |
| SEC22C | NM_001201572 | SEC22 vesicle trafficking protein | 0.0068 | −0.23 |
| | | homolog C (S. cerevisiae) | | |
| NLRC5 | NM_032206 | NLR family, CARD domain | 0.0169 | −0.31 |
| | | containing 5 | | |
| FCER1G | NM_004106 | Fc fragment of IgE, high affinity | 0.0093 | −0.58 |
| | | I, receptor for; gamma | | |
| | | polypeptide | | |
| NTSR1 | NM_002531 | neurotensin receptor 1 (high | 0.0090 | −0.65 |
| | | affinity) | | |

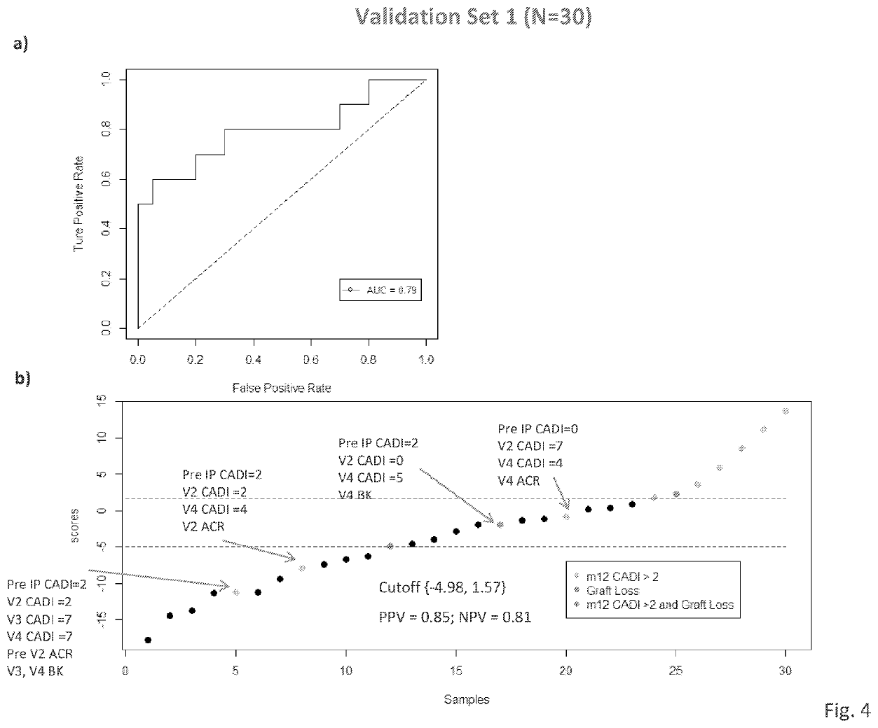

c) To estimate the prediction accuracy: two tertile cutoffs for the gene risk scores (1.57 or −4.98) were defined and demonstrated positive predictive value (PPV)>0.88 and negative predictive value (NPV)=1 for the prediction of fibrosis development in the discovery set (FIG. 3b).
d) Application of the 9 gene set on two validation sets for the prediction of fibrosis: The prediction of early acute rejection by the 9-gene set was validated using V1 dataset (N=30, AUC=0.79, PPV=0.85, NPV=0.81), at the tertile cutoffs defined from the discovery set) (FIG. 4a,b), which is better than the prediction of fibrosis development on V2 with poor pre-implanted kidney or unknown pre-implanted CADI (n=30, AUC=0.58, PPV=0.56 and NPV=0.71)

### EXAMPLE 2: NANOSTRING ASSAY

### Nanostring Assay Kit Will Include

1) Custom CodeSet (barcoded probesets for the 9 gene panel, a housekeeping gene panel and negative controls provided by Nanostring).
2) nCounter® Master Kit including nCounter Cartridge, nCounter Plate Pack and nCounter Prep Pack.
3) QIAGEN RNeasy® Kit for extraction of high quality total RNA.

### Nanostring Experiments

Total RNA will be extracted using the QIAGEN RNeasy® Kit by following the manufacturer's protocol. Barcode probes will be annealed to the total RNA in solution at the point of data collection. After hybridization, the sample will be transferred to the nCounter Pre Station and the probe/target will be immobilized on the nCounter Cartridge. The probes are then counted by the nCounter Digital Analyzer.

### mRNA Transcriptomic Data Analysis

The raw count data from the Nanostring analyzer will be processed in the following procedure: the raw count data will be first normalized to the counts of the house-keeping genes mRNAs and the mRNAs with counts lower than the median plus 3 standard deviations of the counts of the negative controls will be filtered out. Due to data variation arising from reagent lots, the count for each mRNA from different reagent lots will be calibrated by multiplying a factor of the ratio of the averaged counts of the samples on different reagent lots. The calibrated counts from different experimental batches will be further adjusted by the ComBat package.

### EXAMPLE 4: QPCR ASSAY OR QPCR

### The qPCR Assay Kit Includes

1) Primer container (12 tubes with one qPCR assay per tube for each of the 9 genes, which includes the 9 gene-panel and 2 housekeeping genes (ACTB and GAPDH) and the control probe (18S ribosomal RNA). The assays are obtained from LifeTech.
2) TaqMan® Universal Master Mix II: reagents for qPCR reactions
3) TaqMan® ARRAY 96-WELL PLATE (6×23).
4) Agilent AffinityScript QPCR cDNA Synthesis Kit: for the highest efficiency of converting RNA to cDNA and fully optimized for real-time quantitative PCR (QPCR) applications.

### Experimental Procedure and Data Analysis

Total RNA will be extracted from the allograft biopsy samples using the ALLprep kit (QIAGEN-ALLprep kit, Valencia, CA USA). cDNA will be synthesized using the AffinityScript RT kit with oligo dT primers (Agilent Inc. Santa Clara, CA). TaqMan qPCR assays for the 9-gene signature set, 2 housekeeping genes (ACTB, GAPDH) and 18S ribosomal RNA will be purchased from ABI Life Technology (Grand Island, NY). qPCR experiments will be performed on cDNAs using the TAQMAN universal mix and PCR reactions will be monitored and acquired using an ABI7900HT system. Samples will be measured in triplicate. Cycle Time (CT) values for the prediction gene set as well as for the 2 housekeeping genes will be generated. The ACT value of each gene will be computed by subtracting the average CT value for the housekeeping genes from the CT value of each gene.

### EXAMPLE 5: TARGETED RNA SEQUENCING (TREX) ASSAY

### TREx Assay Kit Includes

1) Custom Assay (barcoded probesets for 9 gene panel including a housekeeping gene panel)
2) Illumina® TruSeq® RNA Sample Preparation Kit v2
3) QIAGEN RNeasy® Kit for extraction of high quality total RNA

### TREx Experiments

Total RNA will be extracted using the QIAGEN RNeasy® Kit. The sequencing library will be generated using the Illumina® TruSeq® RNA Sample Preparation Kit v2 by following the manufacturer's protocol: briefly, polyA-containing mRNA will be first purified and fragmented from the total RNA. The first-strand cDNA synthesis will be performed using random hexamer primers and reverse transcriptase followed by the second strand cDNA synthesis. After the endrepair process which converts the overhangs into blunt ended cDNAs, multiple indexing adapters will be added to the end of the double stranded cDNA and PCR will be performed to enrich the targets using the primer pairs specific for the gene panel and housekeeping genes. Finally the indexed libraries will validated, normalized and pooled for sequencing on the MiSEQ sequencer.

### TREx Data Processing

The raw RNAseq data generated by the MiSEQ sequencer will be processed by the following procedure: The reads with good quality will be first aligned to several human reference databases including hg19 human genome, exon, splicing junction and contamination database including ribosome and mitochondria RNA sequences using the BWA alignment algorithm. After filtering reads that mapped to the contamination database, the reads that are uniquely aligned with a maximal 2 mis-match to the desired amplicon (i.e. PCR product from the paired primers) regions will be then counted as expression levels for the corresponding gene and further subjected to quantile normalization across samples after log2 transformation using R statistical programs.

### EXAMPLE 6: HOUSEKEEPING GENE PANEL FOR USE IN THE PRESENT INVENTION

Presented below are 10 housekeeping genes that can be used as gene panels to monitor the quality in the assays of the present invention. 10 genes per panel are used to monitor the quality of the reactions. The kits also contain primers for the housekeeping genes. The 10 housekeeping genes are selected from the group consisting of DERL1, PPID, PRKAG1, PRPF38A, PSMD6, RNF34, RRAGA, TINF2, UBE2G1, UBE2K, USP39 and ZNF394.

A number of embodiments of the methods disclosed herein have been described. Nevertheless, it will be understood that various modifications may be made without departing from the spirit and scope of the invention. Accordingly, other embodiments are within the scope of the following claims.

It is further to be understood that all values are approximate, and are provided for description. Patents, patent applications, publications, product descriptions, and protocols are cited throughout this application, the disclosures of which are incorporated herein by reference in their entireties for all purposes.

1. A method for treating a human renal allograft recipient at risk for developing fibrosis of the allograft comprising the steps of:
(a) selecting a human renal allograft recipient identified as being at risk for fibrosis of the allograft based on detected expression levels of mRNAs encoded by a preselected gene signature set which are higher than the expression levels of a control blood specimen obtained from a second renal allograft recipient that did not develop fibrosis of the allograft, said expression levels obtained by
i. synthesizing cDNA from mRNA isolated from a blood specimen obtained from said renal allograft recipient, and
ii detecting the expression levels of mRNAs encoded by a gene signature set comprising the genes NTSR1, TSPAN14, CCR3, SEC22C, FCER1G, YBEY, CLDN18, NLRC5 and KIAA1683 in the cDNA; and
(b) administering to the selected human renal allograft recipient an effective amount of an anti-fibrotic agent, an anti-rejection drug, or both.

2. The method of claim 1, comprising applying the expression levels determined in the allograft recipient's sample to a penalized logistic regression fitting model to identify the allograft recipient's risk of fibrosis.

3. The method of claim 2, wherein the penalized logistic regression fitting model utilizes the formula:
r=−(log₁₀(p₁)*g₁+log₁₀(p₂)*g₂+ . . . +log₁₀(pᵢ)*gᵢ+ . . . +log₁₀(p₉)*g₉),
where pᵢ is the significance p value of t-test on expression values for gene i (i=1 . . . 9) between the patients with and without development of fibrosis in the training set, gᵢ is a logic number for gene i (i=1 . . . 9), 1 (if the expression value of gene i is greater than the median expression value of the fibrosis group of the training set for an upregulated gene or if the expression value of gene i is less than the median expression value of the fibrosis group of the training set for a downregulated gene), or −1 (if the expression value of gene i is less than the median value of the non-fibrosis group of the training set for an upregulated gene or if the expression value of gene i is greater than the median value of the non-fibrosis group of the training set for a downregulated gene), or 0 (if the expression value of gene i is between the median values of the fibrosis and the non-fibrosis groups of the training set).

4. The method of claim 1, wherein the anti-rejection drug is an immunosuppressive or anti-proliferative agent.

5. The method of claim 4, wherein the anti-rejection drug is sirolimus.

6. The method of claim, wherein the immunosuppressive agent is a member selected from the group consisting of a mycophenolate mofetil (MMF), prednisone, Mycophenolate Sodium and Azathioprine.

7. The method of claim 1, wherein the anti-fibrosis drug is a member selected from the group consisting of Pirfenidone, relaxin, Bone morphogenetic protein 7 (BMP-7) and Hepatic growth factor (HGF) 6.

8. The method of claim 1, wherein detecting the expression levels of said mRNA comprises performing an assay that is a member selected from the group consisting of qPCR analysis, Nanostring analysis and TREx analysis.

9. The method of claim 1, further comprising modifying the immunosuppression regimen of an allograft recipient identified as being at risk for fibrosis of the allograft.

10. The method of claim, wherein modifying the immunosuppression regimen comprises administering to the allograft recipient an effective amount of an anti-rejection drug selected from the group consisting of Belatacept, rapamycin and Mycophenolate Mofetil.

11. The method of claim 10, wherein modifying the immunosuppression regimen comprises administering to the allograft recipient an anti-fibrosis drug selected from the group consisting of Pirfenidone, relaxin, Bone morphogenetic protein 7 (BMP-7) and Hepatic growth factor (HGF) 6.

12. A method for selecting a human renal allograft recipient for treatment to reduce the risk for fibrosis of the allograft comprising the steps of:
(a) detecting expression levels of mRNAs encoded by genes in a gene signature set in a blood sample obtained from the renal allograft recipient, wherein said genes are NTSR1, TSPAN14, CCR3, SEC22C, FCER1G, YBEY, CLDN18, NLRC5 and KIAA1683, and
(b) identifying the human renal allograft recipient as being at risk for fibrosis of the allograft when the expression levels the mRNAs encoded by the genes in the gene signature set are higher than the expression levels of the mRNAs encoded by same genes in a control.

13. The method of claim 12 which comprises determining said expression levels by synthesizing cDNA from mRNA isolated from a blood specimen obtained from said renal allograft recipient, and detecting the mRNA expression levels of the genes in the gene signature set in the cDNA.

14. The method of claim 13, which comprises applying the mRNA expression levels determined in the recipient's blood sample to the penalized logistic regression fitting model.

15. The method of claim 14, which comprises detecting the expression levels of said mRNAs with an assay that is a member selected from the group consisting of qPCR analysis, Nanostring analysis and TREx analysis.

16. A method for identify a renal allograft recipient at risk for fibrosis of the allograft and allograft loss, comprising the steps of
(a) detecting expression levels of mRNAs encoded by a gene signature set comprising the genes NTSR1, TSPAN14, CCR3, SEC22C, FCER1G, YBEY, CLDN18, NLRC5 and KIAA1683 from a blood sample obtained from the renal allograft recipient, and
(b) identifying the human renal allograft recipient as being at risk for fibrosis of the allograft and allograft loss when the expression levels of the mRNAs encoded by the genes in the signature set are higher than the expression levels of the mRNAs encoded by the gene signature set genes in a control.

17. The method of claim 16 comprising administering to the allograft recipient identified as being at risk for fibrosis of the allograft and allograft loss an effective amount of an anti-rejection drug, an immunosuppressive agent, an anti-fibrotic agent or combinations thereof.

18. The method of claim 17 which comprise applying the expression levels determined in the allograft recipient's sample to the penalized logistic regression fitting model to identify the allograft recipient's risk of fibrosis.

19. A method for identify a renal allograft recipient at risk for fibrosis of the allograft and allograft loss, comprising the steps of
(a) detecting expression levels of mRNAs encoded by a gene signature set comprising the genes NTSR1, TSPAN14, CCR3, SEC22C, FCER1G, YBEY, CLDN18, NLRC5 and KIAA1683 from a blood sample obtained from the renal allograft recipient, and
(b) identifying the human renal allograft recipient as being at risk for fibrosis of the allograft and allograft loss when the expression levels of the mRNAs encoded by the gene signature set are higher than the expression levels of the mRNAs encoded by the gene signature set genes in a control.

20. The method of claim 19 comprising administering to the allograft recipient identified as being at risk for fibrosis of the allograft and allograft loss an effective amount of an anti-rejection drug, an immune suppressive agent, an anti-fibrotic agent or combinations thereof.

21. The method of claim 19 which comprise applying the expression levels determined in the allograft recipient's sample to said penalized logistic regression fitting model to identify the allograft recipient's risk of fibrosis.

22. A method for treating a human renal allograft recipient at risk for developing fibrosis of the allograft comprising the steps of:
(a) selecting a human renal allograft recipient identified as being at risk for fibrosis of the allograft based on detected expression levels of mRNAs encoded by a preselected gene signature set which are higher than the mRNA expression levels of the gene signature set in a control, said expression levels obtained by
i. synthesizing cDNA from mRNA isolated from a blood specimen obtained from said renal allograft recipient, and
ii. detecting the expression levels of mRNAs encoded by a gene signature set comprising the genes NTSR1, TSPAN14, CCR3, SEC22C, FCER1G, YBEY, CLDN18, NLRC5 and KIAA1683 in the cDNA; and
(b) administering to the selected human renal allograft recipient an effective amount of an anti-fibrotic agent, an anti-rejection drug, or both.

23. The method of claim 22 wherein the control level is computed based on the mRNA expression levels of the gene signature set.
